(19) United States
(12) Patent Application Publication
(10) Pub. No.: US 2024/0052555 A1
(43) Pub. Date: Feb. 15, 2024

(54) BIOMATERIALS AND BIOTEXTILES AND METHODS FOR MAKING SAME

(71) Applicant: San Diego State University (SDSU) Foundation, dba San Diego State University Research Foundation, San Diego, CA (US)

(72) Inventor: Gregory HOLLAND, San Diego, CA (US)

(21) Appl. No.: 18/267,721

(22) PCT Filed: Dec. 15, 2021
(86) PCT No.: PCT/US2021/063587

§ 371 Date: Jun. 15, 2023

Related U.S. Application Data
(60) Provisional application No. 63/125,612, filed on Dec. 15, 2020.

(51) Int. Cl. D06M 11/05; A61L 15/40; A61L 31/00

(52) U.S. Cl. CPC D06M 11/05; A61L 15/40; A61L 31/005; D06M 2101/12

(57) ABSTRACT

In alternative embodiments, provided are biomaterials comprising hydrated, or hydrated and dried, orb-weaving spider aciniform (AC) prey-wrapping silks, or orb-weaving spider aciniform (AC) prey-wrapping silks that have been substantially converted into β-sheet aggregates or β-sheet nanocrystallite structures, which can be fabricated into products of manufacture, or films, fibers or threads, including fibers or threads woven into textiles or cloths. In alternative embodiments, provided are hydrated, or hydrated and dried, orb-weaving spider aciniform (AC) prey-wrapping silks, or orb-weaving spider aciniform (AC) prey-wrapping silks, substantially comprising β-sheet aggregates or B-sheet nanocrystallite structures, and products of manufacture, or fibers or threads, comprising these β-sheet aggregate-comprising silks or β-sheet nanocrystallite structures. In alternative embodiments, provided are methods for making hydrated, or hydrated and dried, orb-weaving spider aciniform (AC) prey-wrapping silks, or orb-weaving spider aciniform (AC) prey-wrapping silks, substantially comprising β-sheet aggregates or β-sheet nanocrystallite structures.

### RELATED APPLICATIONS

This Patent Convention Treaty (PCT) International Application claims the benefit of priority under 35 U.S.C. § 119(e) of U.S. Provisional Application No. USSN 63/125,612, Dec. 15, 2020. The aforementioned application is expressly incorporated herein by reference in its entirety and for all purposes. All publications, patents, patent applications cited herein are hereby expressly incorporated by reference for all purposes.

### STATEMENT AS TO FEDERALLY SPONSORED RESEARCH

This invention was made with government support under FA9550-17-1-0282 and FA9550-20-1-103 awarded by the US Department of Defense, Air Force Office of Research (DOD-AFOSR), and W911NF2010143 awarded by the Army Research Office (DOD-ARO). The government has certain rights in the invention.

### TECHNICAL FIELD

This invention generally relates to biomaterials and biotechnology. In alternative embodiments, provided are biomaterials comprising hydrated, or hydrated and dried, orb-weaving spider aciniform (AC) prey-wrapping silks, or orb-weaving spider aciniform (AC) prey-wrapping silks that have been substantially converted into β-sheet aggregates or β-sheet nanocrystallite structures, which can be fabricated into products of manufacture, or films, fibers or threads, including fibers or threads woven into textiles or cloths. In alternative embodiments, provided are hydrated, or hydrated and dried, orb-weaving spider aciniform (AC) prey-wrapping silks, or orb-weaving spider aciniform (AC) prey-wrapping silks, substantially comprising β-sheet aggregates and/or β-sheet nanocrystallite structures, and products of manufacture, or fibers or threads, comprising these β-sheet aggregate-comprising silks or β-sheet nanocrystallite structures. In alternative embodiments, provided are methods for making hydrated, or hydrated and dried, orb-weaving spider aciniform (AC) prey-wrapping silks, or orb-weaving spider aciniform (AC) prey-wrapping silks, substantially comprising β-sheet aggregates or β-sheet nanocrystallite structures.

### BACKGROUND

Due to its moderate strength (approximately 700 MPa) and impressive extensibility before breaking (approximately 60-80%), orb-weaving spider aciniform (AC) prey-wrapping silks are actually the toughest of the spider silks but are remarkably understudied. Native AC silk fibers are an α-helix rich coiled-coil/β-sheet hybrid nanofiber, and that conversion of disordered or helical domains to β-sheet aggregates is surprisingly minimal and overall β-sheet content is low (approximately 15%).

### SUMMARY

In alternative embodiments, provided are methods for making a biomaterial comprising: providing an orb-weaving spider aciniform (AC) prey-wrapping silk; and exposing the orb-weaving spider aciniform (AC) prey-wrapping silk to a solvent, an aqueous solution and/or to water to: partially or substantially hydrate the orb-weaving spider aciniform (AC) prey-wrapping silk, and/or, to substantially convert silk alpha helices into silk β-sheet aggregates and/or or β-sheet nanocrystallite comprising structures.

In alternative embodiments, of method as provided herein:
the solvent comprises a water-miscible protonic and/or a polar aprotonic organic solvent, and optionally the solvent comprises an alcohol,

and optionally the solvent comprises an alcohol or a glycol, and optionally the alcohol or glycol comprises ethylene glycol, methanol, propanol, butanol, isopropanol, isobutanol, furfuryl alcohol, 1,2-butanediol, 1,3-butanediol, 1,4-butanediol, 2-butoxyethanol, and/or hexafluoroisopropanol,

and optionally the solvent comprises an acid or is an acid solution, and optionally the acid comprises phosphoric acid, formic acid, butyric acid, trinitrobenzenesulfonic acid (TNBS) and/or acetic acid,

and optionally the solvent comprises an inorganic salt, and optionally the inorganic salt comprises a halide and/or a thiocyanate,

and optionally the solvent comprises dimethyl sulfoxide, 1, 4 dioxane, ethylamine, N-methyl-2-pyrrolidone, glycerol, acetone, acetaldehyde, diethanolamine, diethylenetriamine, dimethylformamide, dimethoxyethane, triethylene glycol, tetrahydrofuran, and/or acetonitrile;
approximately 1% to 99%, or 5% to 90%, or 10% to 80%, or 20% to 70%, or at least 5%, 10%, 15%, 20%, or 55%, by weight of the hydrated orb-weaving spider aciniform (AC) prey-wrapping silk is the water or aqueous solution and/or solvent;
the orb-weaving spider aciniform (AC) prey-wrapping silk is water-wetted or hydrated and/or solvated (or solvent wetted) for between about 10 minute to 10 hours or more, or for about 2 hours (hr);
the hydrated orb-weaving spider aciniform (AC) prey-wrapping silk is dried, optionally the hydrated or solvated orb-weaving spider aciniform (AC) prey-wrapping silk is dried for between about 1 hour and 5 days, or for between about 10 hours and 3 days, or more, or for about 2, 3, 4, 5, 6 or more days;
the substantially or partially dried orb-weaving spider aciniform (AC) prey-wrapping silk has a substantially β-sheet nanocrystallite structure; and/or
approximately 5% to 99%, or 10% to 90%, or 15% to 85% or 20% to 80%, or at least about 80%, 85%, 90%, 91%, 92%, 93%, 94%, 95%, 96%, 97%, 98%, or 99%, or more of the orb-weaving spider aciniform (AC) prey-wrapping silk alpha helices are converted into silk β-sheet aggregates or β-sheet nanocrystallite structures.

In alternative embodiments, provided are compositions or products of manufacture comprising a biomaterial made by a method as provided herein; and optionally the composition or a product of manufacture comprises or is fabricated as a film, thread or fiber, and optionally the thread or fiber is woven or fabricated into a cloth, textile or a fabric.

In alternative embodiments, provided are compositions or products of manufacture comprising a plurality of silk β-sheet aggregates or β-sheet nanocrystallite structures, made by process comprising a method as provided herein.

In alternative embodiments, provided are products of manufacture comprising a biomaterial comprising orb-weaving spider aciniform (AC) prey-wrapping silk, wherein approximately 5% to 99%, or 10% to 90%, or 15% to 85% or 20% to 80%, or at least about 85%, 90%, 91%, 92%, 93%, 94%, 95%, 96%, 97%, 98%, or 99%, or more of the orb-weaving spider aciniform (AC) prey-wrapping silk alpha helices are converted into silk β-sheet aggregates or β-sheet nanocrystallite structures; and optionally the products of manufacture are fabricated as a film, thread or fiber, and optionally the film, thread or fiber is woven, spun or fabricated into a cloth, textile or a fabric or any wearable material (including hats, boots, gloves, helmet, body armor, masks), or optionally fabricated as a container or a medical device, optionally a stent, or a bandage. In alternative embodiments the silk β-sheet aggregates or β-sheet nanocrystallite structures are incorporated into a plastic, a wood, a synthetic material, or a cloth or any wearable material or item, including hats, helmets, gloves, masks, shoes, clothes, space suits, body armor, and the like. In alternative embodiments the silk β-sheet aggregates or β-sheet nanocrystallite structures are incorporated into, or fabricated, as a structural material, for example a structural and/or building material of an automobile, an airplane, a spacecraft, a boat, a dwelling or office or commercial building, including for example, for use or incorporation into or as walls, ceilings or roofs, insulating materials, lining materials, ducts, piping, wiring insulation, wall or ceiling coverings, panels, sinks, or floors.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1 schematically illustrates Argiope argentata AcSp1 prey wrapping silk; in the A. argentata species, there are 20 iterated repeats of the wrapping (“W”) subunit; the solution Nuclear Magnetic Resonance (NMR) structure from a single A. trifaciata W subunit (PDB 2MU3) was used to create the theorized “beads-on-a-string” model hypothesized to occur in solution and colored to identify each area; the colored sequences are for A. argentata AcSp1:

Helix 1:
(SEQ ID NO: 1)
AGPQGGFGAT GGASAGLISR VANALANTST LRTVLRTGVS
QQIASSVQRA AQSLASTLG
Helix 2:
(SEQ ID NO: 2)
VDGNNLARFA VQAVSRL
Helix 3:
(SEQ ID NO: 3)
PAGSDTSAYA QAFSSALFNA GVL
Helix 4:
(SEQ ID NO: 4)
NASNIDTLGS RVLSALLNGV SSAAQGLG
Helix 5:
(SEQ ID NO: 5)
INVDSGSVQS DISSSSSFL
Linker/string:
(SEQ ID NO: 6)
STSSSSASYS QASASSTSGS GYTGPSGPST
GPGYPGPLGG GAPFGQSGFG G.

FIG. 2 illustrates images of: (a), A. argentata spinning a cricket with AC silk; (b), the same spider returning to eat its prey through EOD; (c), SEM images comparing prey-wrapping silk fibers from A. argentata after feeding or water treatment.

FIG. 3 graphically illustrates ¹³C Cross-Polarization Magic-Angle-Spinning (CP-MAS) Solid-State Nuclear Magnetic Resonance (SSNMR) spectra of A. argentata AC silk; (a), AC silk labeled with ¹³C-Ala; (b), AC silk labeled with ¹³C-Val. Peaks in (a) with asterisks are from crystalline Ala contamination.

FIG. 4 graphically illustrates: (a), 2D ¹³C-¹³C dipolar-assisted rotational resonance (DARR) SSNMR spectra collected with a 100 millisecond (ms) mixing time for ¹³C-Ala-labeled A. argentata AC silk after different treatments; (b), the same as in (a) but only ¹³C-Val labeled; (c), spectral deconvolutions of 1D ¹³C CP-MAS data showing qualitative changes to the Ala Cβ (left) and Val Cβ (right) resonance after different treatments; the fit parameters are summarized in Table 1.

FIG. 5 graphically illustrates: (a), Example of 2D ¹H/¹³C WISE spectrum with a 50 ms spin-diffusion time for hydrated A. argentata AC silk; (b), deconvoluted fits of ¹H slices extracted at the Ala Cα β-sheet resonance in the WISE spectrum for AC silk and BW dragline; (c), build-up curves of narrow component (red) from deconvolutions for the two silks; the arrows indicate th for each sample.

FIG. 6 schematically illustrates an exemplary model of AC silks after contact with water or solvent; helices 1-5 are blue and green cylinders that form α-helical coiled-coils: (a), before wetting there is little β-sheet content (approximately 15%); (b), after water or solvent wetting some α-helical and RC structures convert to β-sheet, illustrated as shortened coiled-coils and the multi-colored arrows colored according to their origin from RC and/or helix structures.

FIG. 7 (or Figure S1) illustrates images of AFM topography scans of (a) native prey-wrapping silk and (b) silk water-wetted for 2 hours (hr) and dried for 2 days.

FIG. 8 (or Figure S2) graphically illustrates: ¹H slice extracted at Ala Cα β-sheet resonance in WISE spectra collected with 50 ms spin-diffusion mixing times for the two silks. Clearly the AC silks exhibit larger narrow component indicating mobile magnetization is able to spin diffuse into Ala β-sheet domains more rapidly because of smaller nanocrystallite dimensions compared to Black Widow (L. hesperus) dragline MA fibers.

FIG. 9 (or Figure S3) graphically illustrates: WISE build-up curves for A. argentata AC and Black Widow (L. hesperus) MA silk fibers. The first four points of the initial Gly build-up are fit with a dashed line of the corresponding color and the reported buildup time (\sqrt{τ*ₘ}) is listed next to the label. Spin-diffusion occurs rapidly into the Ala Cα and Gly Cα areas of AC silks suggesting they are smaller than in the BW silk fibers. Table S2 has these values.

FIG. 10 (or Figure S4) graphically illustrates: thermogravimetric analysis (TGA) of A. argentata AC silks; Native silks were collected and run within 5 minutes of collection; Water loss is reported in Table S3 from the weight loss curve up to 120° C.; The data was collected with a TA2910 (TA Instruments Inc.) instrument under a steady nitrogen flow (60 mL/min for furnace and 40 mL/min for balance); Prior to analysis, the sample was kept under N2 flow for 30 minutes to remove weakly-bound, physiosorbed water and obtain a stable baseline.

FIG. 11 illustrates Table 2, a summary of isotopically labeled AC spider silks for SSNMR experiments, how the material was treated, corresponding observed and our interpretation.

FIG. 12 schematically illustrates an exemplary process for making wet-shearing or solvent-shearing AC silk materials as provided herein, comprising use of a Magic-Angle-Spinning (MAS) rotor within an NMR probe head for wet-shearing or solvent-shearing AC silk materials; NMR spectroscopy can be conducted during the process to determine structural changes that occur to the silk due to mechanical wet-shearing or solvent-shearing, as described in detail in Example 2, below.

Like reference symbols in the various drawings indicate like elements.

### DETAILED DESCRIPTION

In alternative embodiments, provided are biomaterials comprising hydrated, or hydrated and dried, orb-weaving spider aciniform (AC) prey-wrapping silks, or orb-weaving spider aciniform (AC) prey-wrapping silks, that have been substantially converted into β-sheet aggregates and/or β-sheet nanocrystallite structures, which can be fabricated into products of manufacture, including for example, films, fibers or threads, including films, fibers or threads woven into textiles or cloths, or any wearable material (masks, gloves, helmets, shoes, boots), insulating materials, adsorbant or absorbant materials, storage or container materials such as for example, for fabricating mailing envelops, boxes or cartons, or for medical devices or bandages, or for any consumer product with or in place of a plastic material.

In alternative embodiments, products of manufacture as provided herein comprise or are fabricated as a chord, a film, a thread or a fiber, or a film formal, and optionally the chord, film, thread or fiber is woven or fabricated into a cloth, textile or a fabric, or any wearable material, or any manufactured flexible or malleable material. In alternative embodiments, products of manufacture as provided herein are fabricated or manufactured as a cloth, a textile or a fabric or any biomaterial, where optionally the products of manufacture are malleable and/or biodegradable.

### Wet-Shearing or Solvent-Shearing

In alternative embodiments, provided are biomaterials comprising hydrated, or hydrated and dried (using for example water and/or a solvent), orb-weaving spider aciniform (AC) prey-wrapping silks, or orb-weaving spider aciniform (AC) prey-wrapping silks, that have been substantially converted into β-sheet aggregates and/or β-sheet nanocrystallite structures, are made or fabricate by a process comprising use of a wet-shearing or solvent-shearing method as provided herein, as described in detail in Example 2, below.

In alternative embodiments, exemplary processes for wet-shearing AC silk fibers comprise:
(1) soaking or incubating a silk or silk sample in water (H₂O) or solvent to substantially hydrate or solvate, for example, the silk or silk sample is soaked for minutes (mins) to days, optionally between about 4 minutes to 60 minutes, or between about 1 hour to 24 hours, or between about a day to one week, or a week or more,
(2) removing excess water or solvent, for example, with evaporation or an adsorbant or an absorbant material, such as a KIMWIPE™ or equivalent, wherein optionally between about 50% to 99% of the water and/or solvent is removed, or at least about 70%, 75%, 80%, 85%, 90%, 91%, 92%, 93%, 94%, 95%, 96%, 97%, 98%, or 99%, or more of the water and/or solvent is removed, thereby producing a “wet” or “solvated” silk sample or material, and
(3) controlling the pH of the water and/or solvent with an acid or a base having a pH of between about pH 1 and 14, and or controlling the temperature to between about 0° ° C.and 100° C.;
(4) moving or transferring the “wet” or “solvated” silk sample to a magic angle spinning (MAS) solid-state nuclear magnetic resonance (NMR) rotor for mechanical wet-shearing, optionally using protocols described for example, in Andrew, E. R., et al., Nuclear Magnetic Resonance Spectra from a Crystal Rotated at High Speed. Nature 1958, vol. 182, pg 1659; Zhang, R., et al., Proton-based Ultrafast Magic Angle Spinning Solid-state NMR Spectroscopy. Acc. Chem. Res. 2017, vol. 50, pg 1105.

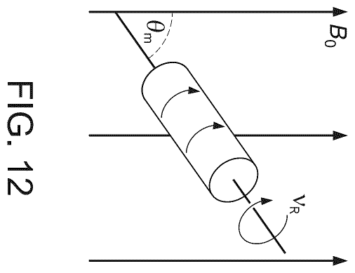

In alternative embodiments, the MAS rotor is spun about the magic angle (54.7°) with respect to the NMR spectrometer external magnetic field, B₀ (see for example, FIG. 12).

In alternative embodiments, the MAS speed or rotor frequency, ν_{R} (Hz; s⁻¹), is precisely controlled with a MAS pneumatic spin control unit (for example, a MAS pneumatic spin control unit Bruker Biospin). Various rotor diameters can be used that dictate the maximum rotor frequency and can range from between about 1 KHz (kilohertz) to about 100 KHz, which depends on the rotor diameter. Rotor diameters can be varied from between about 0.5 mm to about 10 mm, and the spinning speed can be controlled to about less than (<) 1%. In alternative embodiments, the MAS NMR probe has a temperature control unit to precisely control sample temperature, for example, to any setting between about 0° C. and 100° C.

In alternative embodiments, although water is described as the wetting solvent for the silk in the exemplary process provided herein, it does not need to be, and different solvents can be used, for example, any solvent can be used, for example, any solvent that can have an impact on the degree of shearing and the molecular structure of the final processed silk product and resulting mechanical properties, for example, an alcohol or an organic solvent, for example any water-miscible protonic and polar aprotonic organic solvent or mixture thereof can be used.

In alternative embodiments, soaking time in H₂O or other solvent, length of time under MAS (degree of shearing), ν_{R}, and temperature are varied to impact the structure and mechanical properties of the final processed AC silk material.

The act of dehydrating (drying) the solvent-soaked silk under shear controls the final material structure (β-sheet content) and mechanical properties (stiffness). This method of wet-shearing the silk within a MAS NMR probe allows for determination of changes in the molecular structure (such as for example, protein-folding) of the silk during processing via MAS NMR spectroscopy. The act of wet-shearing or solvent-shearing hydrated AC silk results in a substantial increase in β-sheet content which impacts the mechanical properties of the material.

Any of the above aspects and embodiments can be combined with any other aspect or embodiment as disclosed here in the Summary, figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: Making Exemplary Biomaterials

This example demonstrates exemplary methods for making hydrated, or hydrated and dried, orb-weaving spider aciniform (AC) prey-wrapping silks, or orb-weaving spider aciniform (AC) prey-wrapping silks that have been substantially converted into β-sheet aggregates or β-sheet nanocrystallite structures, and products of manufacture comprising same.

We demonstrated through Scanning Electron Microscopy (SEM) that native orb-weaving spider aciniform (AC) silk fibers undergo matted cross-linking upon exposure to moisture. The unique molecular mechanism of water-induced cross-linking is revealed with solid-state NMR (ssNMR) methods; water-induced morphological changes are correlated with an increase in AC silk protein β-sheet content, and additionally we observe a minor unfolding of coiled-coil regions. Continued and increased β-sheet cross-linking are observed upon application of mechanical shear.

We determined the size of these formed β-sheet regions to be between 3 to 7 nm using WISE SSNMR. The observation that merely water treatment can be used to convert a protein-based material from a flexible/extensible α-helix-rich coiled-coil fiber to a rigid crossed-linked β-sheet mat is an entirely new observation providing new avenues in bioinspired materials design.

The mysteries of spider silks have intrigued researchers and inspired polymer engineers for decades due to their impressive and wide ranging mechanical properties, their biodegradability and biocompatibility, and the fact that nature's superfibers are spun on-demand at ambient temperature and pressure from an aqueous silk dope.^{1,2} It has been proposed that spider silks could be a suitable material for protective clothing, parachutes and cables, wound treatment, tissue scaffolding, and medical implants.³⁻⁵ Spiders are unfortunately territorial creatures and therefore domestication and silk harvesting is not a practical strategy to bring the material to market. Artificial production and utilization of spidroin-based recombinant proteins are therefore the only alternative. Imperative to attaining this goal is a more complete understanding of the molecular structure, function, storage mechanism and spinning process of the native material. Of the seven types of silks and glues produced by orb-weaving spiders, Aciniform (AC) prey-wrapping spider silks, utilized for prey-wrapping and egg-case linings, are the toughest of the spider silks, but surprisingly little information exists on their molecular structure and detailed structure-function relationship.⁶⁻⁸ Remarkably, AC silks boast mechanical properties that weight for weight surpass the toughest natural and man-made materials, including Kevlar®, high-tensile steel and even the most studied spider silk, dragline silk.^{6,9} Due to their moderate (approximately 700 MPa) mean ultimate strength and high (approximately 60-80%) extensibility before breaking, AC silks are reportedly 50% tougher than the highly-coveted spider dragline fibers.^{9,10}

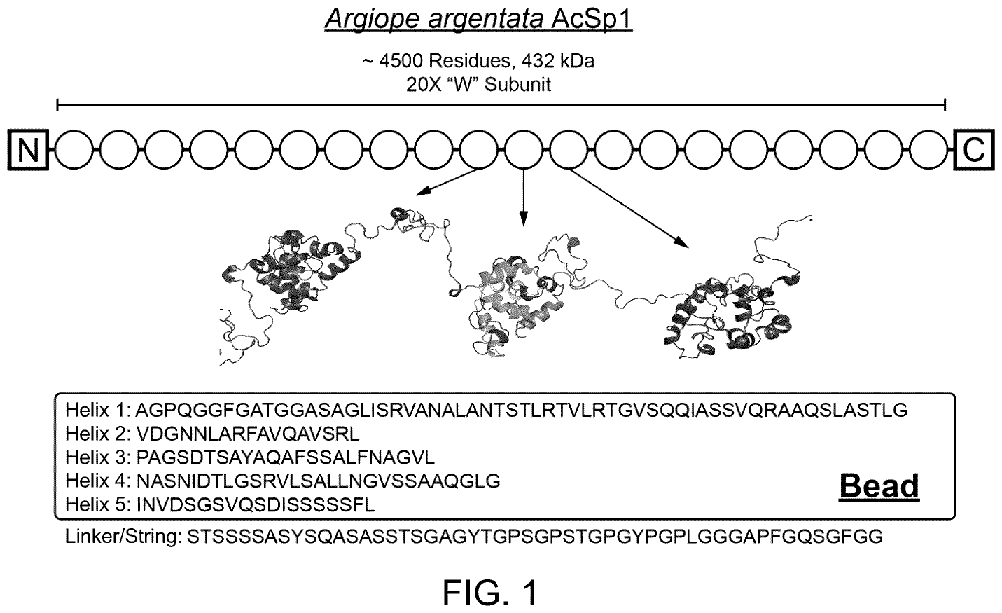

Generally, silk protein biopolymers are composed of highly repetitive, high molecular weight (MW) proteins (about 250 to 500 kDa) rich in small-sidechain amino acids Ala, Gly (approximately 60% to 75%) and to a variable extent Gln, Ser, Pro, Tyr and Arg.^{11,12} The major ampullate (MA) silk proteins that comprise dragline silk are highly dynamic, intrinsically disordered proteins (IDPs) as stored in the MA silk gland prior to fiber spinning.¹³ In the final spun fibers, the poly(Ala)ₙ (n=4 to 9) and flanking (Gly-Ala) repeats assemble into β-sheet nanocrystallites (approximately 2 to 7 nm)¹⁴⁻¹⁷ dispersed throughout a Gly-Gly-X (X=Gln, Tyr, Pro, Ser and Arg)-rich matrix that is non-β-sheet and best described as a disordered helix that is not alpha-helical.^{18,19} Aciniform spidroin 1 (AcSp1), the primary protein making up spider wrapping silk, is markedly different than dragline silk and is composed of a string of 14 to 20 identical subunits (depending on species) of about 200 amino acids (called a W unit for wrapping), flanked by non-repetitive N- and C-terminal regions (FIG. 1).²⁰ Solution-state NMR studies on a single recombinantly produced AcSp1 wrapping unit suggest that each 200 amino acid W subunit folds into a multi-domain structure composed of a well-defined helix-rich globular domain and a glycine/serine-rich disordered linker region.²¹⁻²⁴ It was proposed that each W subunit forms a “beads on a string” model when the protein is stored in the gland.²² Synthetic versions of AC silk fibers have previously been generated from an aqueous dope in order to study the mechanical properties imparted by different buffers as well as differences in the number of W subunits.²⁵⁻²⁷ Although recombinant AcSp1 W subunits have been well characterized as recombinant protein constructs in a solubilized state, a molecular-level understanding of the native AC silk protein after conversion to insoluble fiber is quite minimal. Recently, we applied Solid-State Nuclear Magnetic Resonance (SSNMR) techniques to isotopically-enriched native prey-wrapping silks and quantitatively correlated the secondary protein structures of Ala, Ser and Val residues to the primary protein sequence of the W subunit.

We proposed a hybrid alpha-helical coiled-coil/β-sheet hierarchical model for A. argentata and A. aurantia spider prey-wrapping silk fibers, in which 1) the globular helix-rich domains (bead) mostly retain their helical structures in the final fiber, 2) where the Ala/Ser-rich motifs from the string/linker region aggregate into β-sheet nanostructures, and 3) a significant fraction of the protein remains disordered.²⁸ We intuitively argued that the high extensibility of AC silk fibers is a direct result of high α-helical content, while its moderate mean ultimate strength arises from β-sheet nanostructures and possibly coiled-coil interactions between fiber-aligned helices.

It is likely that when stored in the AC gland, the silk protein is highly helical with no evidence of β-sheet secondary structure, but there is clear evidence of a minor population of β-sheet aggregates in the final fiber.²⁸⁻³⁰ Hence, a structural conversion from either RC and/or α-helical regions into β-sheet nanostructures takes place upon fibrilization. There is some debate over the precise mechanism. For example, Raman studies suggest that wrapping silks from N. clavipes are roughly 50% helical in the gland and 24% helical and 30% β-sheet in the fiber,^{29,30} which are in poor agreement with our recent results where we determine the native fiber is approximately 50% helical and approximately 15% β-sheet, albeit from a different spider species.²⁸ Additionally, solution-state NMR data on A. trifaciata recombinant W subunits show higher dynamics of the Ser-rich stretch in the globular domain, denoted helix 5 (FIG. 1), and the authors suggest that the Ser-rich helix 5 motif is a likely candidate for α-helix to β-sheet aggregation as a driving event for fibrilization.^{22,24} However, this again conflicts with our SSNMR results, namely that in order for Ala, Val and Ser secondary structures to all agree with SSNMR observations, the Ser-rich region in the helix 5 domain must remain helical or loosely-helical but not β-sheet in the fiber.²⁸ Perhaps the true fibril protein structure is somewhere in between, existing as heterogeneous structures with incomplete conversion of poly(Ser) and poly(Ala-Ser) motifs from RC and/or α-helices in the gland to β-sheet structures in the final fiber. As such, there are clearly regions of the primary protein sequence that may be primed to β-sheet aggregate if given the appropriate conditions.

Herein is provided and described further molecular and nanoscale structural insights into native AC silk fibers and demonstrate that AC silks exhibit a remarkable β-sheet fiber cross-linking property when the silk is exposed to water that has not been observed in any other silk to the best of our knowledge. Remarkably, single approximately 500 nm wrapping fibers are visibly cross-linked into fibrous sheets and mats when exposed to water. SSNMR results on native and water-induced cross-linked fibers illustrate that disordered linker regions and α-helical structures decrease with a corresponding increase in β-sheet content for Ala, Ser, Gly, Gln and Val residues. This suggests that the linker/string environments and loosely-structured helices are most sensitive to water-induced β-sheet crosslinking. When mechanical stress is applied to hydrated silks, the data shows a further increase in β-sheet structures and a corresponding reduction in α-helical and random coil content. While the invention as provided herein is not limited by any particular mechanism of action, we hypothesize that exposure to moisture provides disordered and semi-stable helical regions of the wrapping silk with the necessary degrees of freedom to access lower-energy β-sheet aggregates. We think this remarkable moisture-induced cross-linking behavior is an evolutionarily adapted trait that benefits the spider during Extra-Oral Digestion (EOD) and feeding, and should be of great interest to the synthetic biomimetic materials community; converting a malleable and extensible fibers into rigid crossed-linked biomaterial simply by hydrating with water and drying.

### RESULTS AND DISCUSSION

### Water-Induced Crosslinking of Native AC Spider Silk Nanofibers

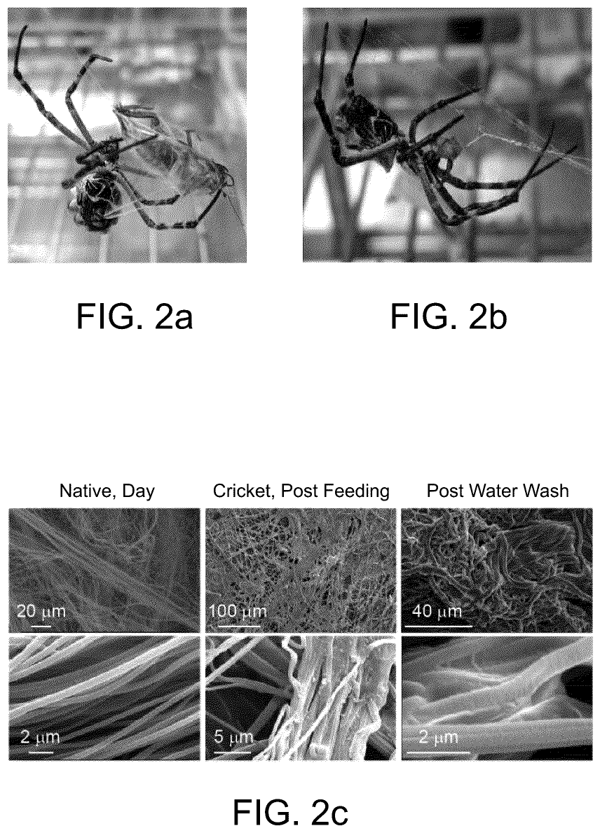

Freshly-collected AC silk is shown as a dense mesh of fine (approximately 500 nm in diameter) fibers (FIG. 2c). When we attempted to image silk on a wrapped and consumed prey (cricket), we were surprised to observe clear morphological changes to regions of the silk. Dense bundles of individual AC fibers remain, but areas of wrapping silk fibers appear to have fused together into fibrous sheets or mats, especially in regions where the silk makes direct contact with the cricket. Individual fibers clearly have fused together to form larger, uniaxial fibers and fibrous mats. Additionally, inter-fiber space appears to have collapsed. To determine the effect of water on AC fiber morphology, native prey-wrapping silk was briefly soaked in deionized water for two hours without mechanical agitation. SEM images of our water-wetted silk bundles confirm that this observed cross-linking property is water-induced. After allowed to air-dry the material became noticeably stiffer and was no longer malleable like the native silk fiber. It is possible that other factors, such as dissolved salts and pH used in the native (wrapped cricket) fibers, may fine tune the local silk fusion and aggregation to minimize fluid loss from the prey. The appearance of nearly identical morphological structures in both native and water-treated samples, however, leads us to the conclusion that AC silk protein-water interactions drive the mechanism for fiber fusion and cross-linking.

### Solid-State NMR Characterization of Hydration-Induced Structural Changes.

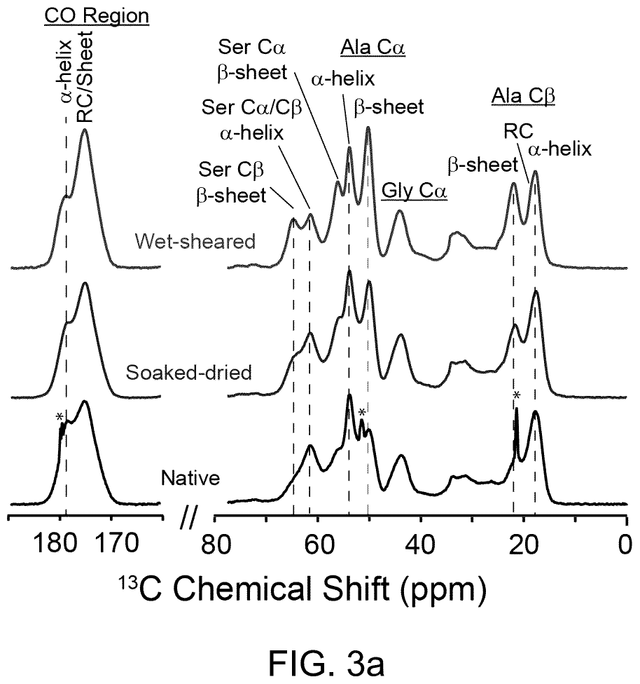

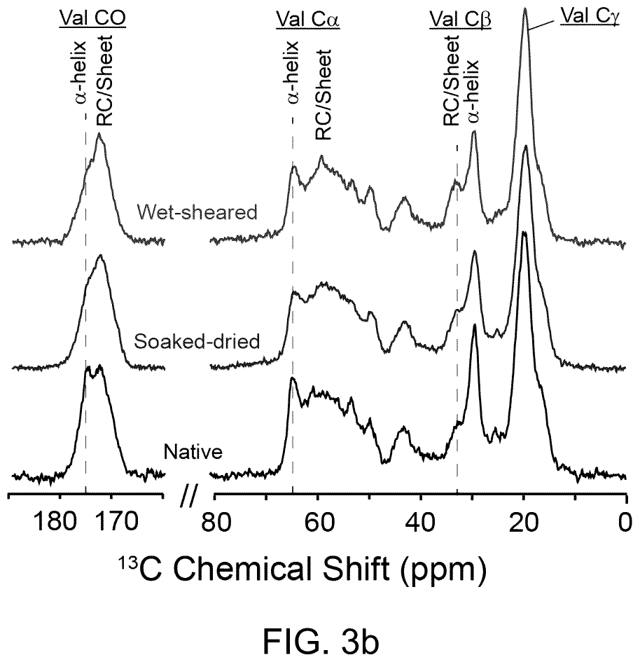

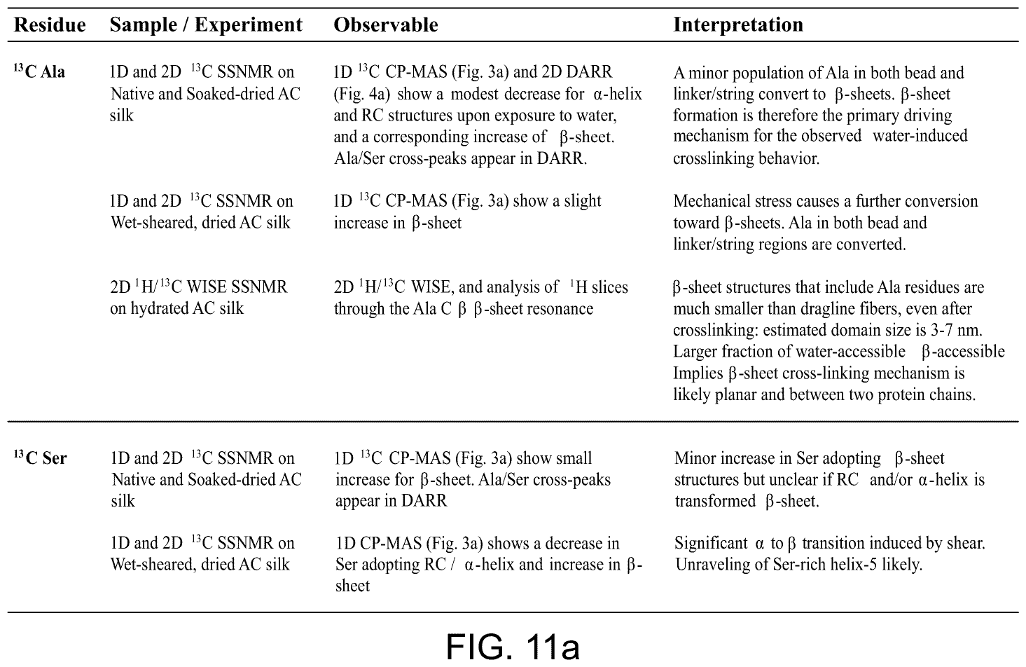

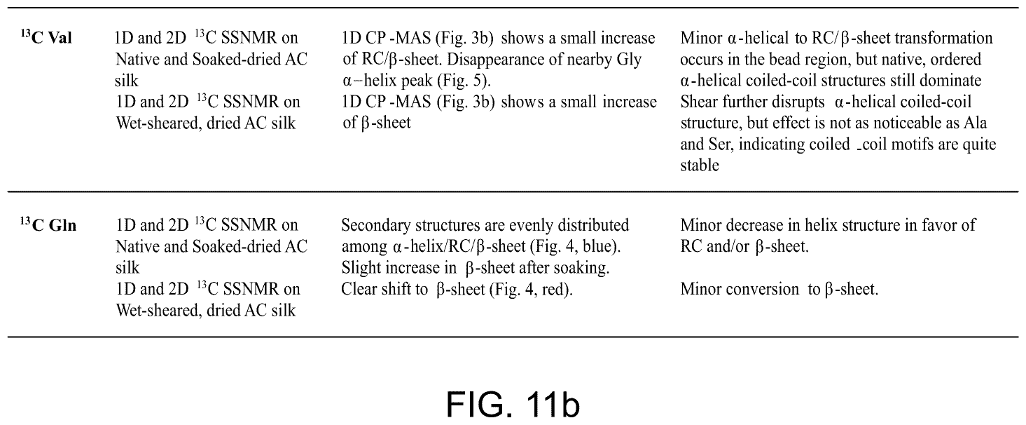

The likely biological reasoning behind the observed phenomenon is that Argiope spiders, including the A. argentata species in this study, will first immobilize their prey using wrapping silk in an act called “attack wrapping” (FIG. 2a), then bite to inject their venom.^{31,32} After paralyzing their prey, they then engage in EOD, whereby they digest their food outside of their bodies by regurgitating digestive fluids into or onto the prey to consume them, during which prey-package manipulation is common adding a degree of mechanical stimulation through physical agitation and rotation (FIG. 2a).³³ Therefore, to better understand the structural changes induced by both moisture and mechanical agitation, SSNMR data was collected on AC silks after two types of sequential treatment. After initial SSNMR analysis on the native fibers (“Native”, FIG. 3 black), the AC silk was removed from the NMR rotor and soaked with DI water at room temperature for two hours without any mechanical agitation and allowed to air-dry (“Soaked-dried”, FIG. 3 blue). After data collection, the AC silk was re-hydrated and loaded into the rotor while still wet which allows the spinning rotor to induce physical shear, removed and dried for two days, then reloaded. (“Wet-sheared, dried”, FIG. 3 red). The changes to the protein secondary structure for several amino acids found throughout the W subunit were monitored throughout this process and a summary of these conformational changes (Table 1) and our interpretation are listed in Table 2 (see FIG. 11).

### Alanine

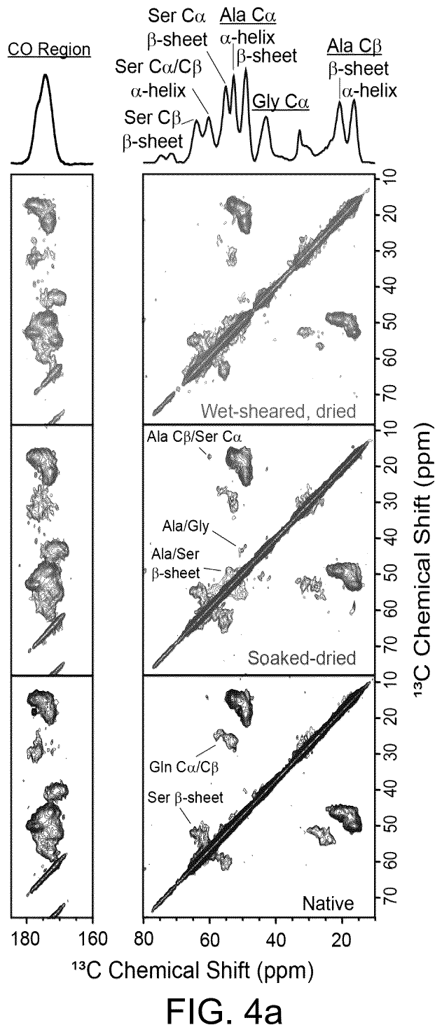

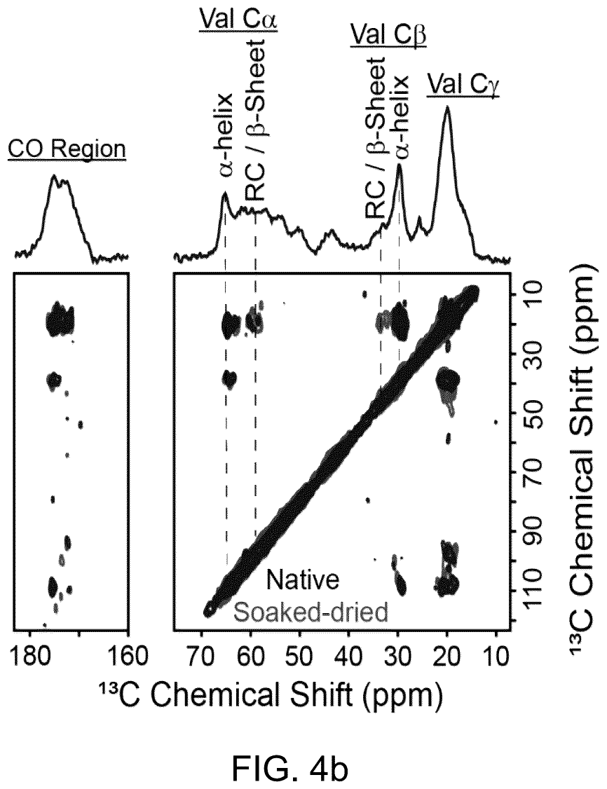

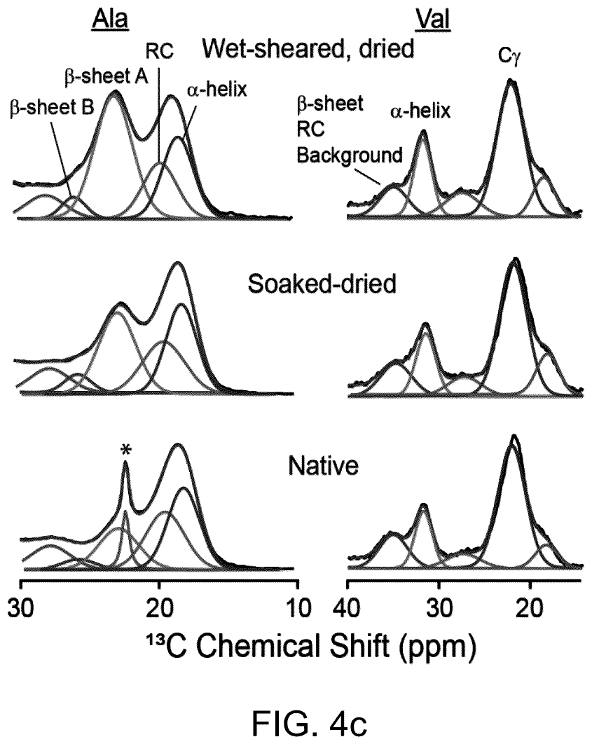

Several conclusions can be drawn from comparing 1D ¹H/¹³C cross polarization magic angle spinning (CP-MAS) spectra between the native and water-treated silk (FIG. 3a). When scaling the 1D ¹³C-Ala-labeled CP-MAS spectra based on the Gly Cα resonance, it becomes clear that Ala β-sheet content increases moderately upon water wetting (FIG. 3a). The reader is reminded that this sample was only submerged in water for two hours with no agitation and allowed to air-dry prior to data collection, thus these structural changes are truly a result of exposure to water and not from shearing or mechanical stress caused by magic angle spinning (MAS). It is not immediately clear if this observed structural transition is due to unfolding of α-helical environments in favor of β-sheets, or if previously disordered regions of the silk protein are enticed to aggregate. Insight is gained through spectral deconvolutions of the data. To understand water-induced structural changes we first extracted precise chemical shifts from 2D ¹³C-¹³C DARR spectra (FIG. 4a), and then peak-fit the 1D ¹³C CP-MAS spectra using chemical shifts and estimated linewidths extracted from the 2D spectrum (FIG. 4c). Two peaks are required to fit the Ala Cβ β-sheet resonance because of different side-chain packing arrangements that have been proposed from studies on silk-like model peptides.³⁴ This procedure, outlined in the supplemental section of our prior work, was applied.²⁸ These results show that the β-sheet content for Ala increases from approximately 30% for the native silk to approximately 41% after wetting, and reaches a maximum of approximately 53% after wet-shear and drying (FIG. 3a, Table 1). As a comparison, Ala within poly(Ala)-rich spider dragline silks show much higher β-sheet content at 82%.³⁵ This increase in β-sheet content for AC silks is mirrored by a decrease of Ala in RC structures, followed by α-helical structures after shear (Table 1). Ala occupies α-helical, RC and β-sheet structures in approximately equal proportions after wetting. After wet-shearing and drying, Ala adopting α-helical and RC structures further decreases while β-sheet content increases to nearly half. The solution structure of the W subunit shows that helix 1 contains 11 of the 26 Ala residues, where the linker contains five.²⁰ Therefore, this data implies that the most likely Ala conversion comes from the partially-coiled helix 1 and linker regions.

TABLE 1
Summary of conformation dependent ¹³C isotropic chemical shifts
and secondary structure populations determined from peak deconvolutions
of ¹³C CP-MAS spectra for AC silks (FIG. 4).

| | Chemical Shift* | | | | | | Normalized Peak Area | | |
|---|---|---|---|---|---|---|---|---|---|
| Residue | (ppm) | | | Peak Width (ppm) | | | (%) | | |
| Ala Cβ | Native | SD | WSD | Native | SD | WSD | Native | SD | WSD |
| α-helix | 18.2 | 18.3 | 18.3 | 2.7 | 2.7 | 2.6 | 38.0 | 32.7 | 26.5 |
| Random Coil | 19.6 | 19.6 | 19.5 | 3.3 | 3.7 | 2.9 | 32.9 | 26.1 | 19.9 |
| β-sheet A | 22.9 | 22.9 | 22.8 | 3.4 | 3.2 | 3.2 | 24.0 | 35.6 | 48.1 |
| β-sheet B | 25.8 | 25.8 | 25.8 | 2.8 | 2.3 | 2.1 | 5.1 | 5.6 | 5.5 |
| Val Cβ | | | | | | | | | |
| α-helix | 31.6 | 31.6 | 31.7 | 1.9 | 1.9 | 1.9 | 62.0 | 54.4 | 50.9 |
| RC/β-sheet/ | 34.8 | 34.8 | 35.0 | 3.1 | 3.1 | 3.1 | 38.0 | 45.6 | 49.1 |
| Background | | | | | | | | | |

SD: Soaked-dried,
WSD: Wet-sheared, dried
*from DSS ³⁶

### Serine

Similar to Ala, there is a clear but minor increase in Ser adopting β-sheet structures after exposure to water. The relative intensity of the Ser Cα and Cβ -sheet resonances at 55 and 65 ppm increase slightly upon exposure to water, and then increase dramatically after wet-shearing (FIG. 3a). Unfortunately, due to the high spectral overlap in that region, we cannot confidently determine if this is from a partial disruption of α-helical structures or a collapse of disordered regions into β-sheet nanostructures. In our previous model of native AC silks, we demonstrated that the poly(Ser) motif at the end of helix 5 remains helical in the final fiber.²⁸ This is intriguing, since poly(Ser) motifs found in other insect silks have been shown to form β-sheet structures.^{37,38} While a partial α to β conversion of this poly(Ser) helix 5 may occur during water-wetting, the surprisingly low conversion towards β-sheets with only soaking suggests that a full conversion does not occur. After wet-shearing and drying, we see a more substantial decrease in Ser α-helix content in favor of β-sheet structures, implying collapse of the poly(Ser) helical motif.

### Valine

1D ¹³C CP-MAS data for ¹³C-Val enriched AC silk before and after water-washing suggests that Val helical structures partially collapse from 62% to approximately 51% (FIG. 3b, Table 1), where broad and weak Val Cα/Cγ and Val Cα/Cγ cross-peaks in the 2D DARR become slightly stronger (FIG. 4b). This qualitative observation is supported by spectral deconvolutions of the Val Cβ signal, although we could not confidently separate β-sheet and RC structures (FIG. 4c, Table 1). Since Val residues are almost exclusively found in the globular “bead” region and adopt coiled-coil like superstructures in the final fiber, it is likely that a minor conversion of Val from helical to RC and/or β-sheet motifs occurs in this region upon exposure to water. One explanation for this conversion is that several of the Val residues in the “bead” region are found as Gly-Val/Val-Gly pairs. Perhaps the juxtaposition of Gly and Val create a more favorable RC or β-sheet environment during water-wetting, since Gly is known to be unstructured in many proteins. Indeed, closer inspection of the Gly peak in the Ala-labeled native AC silk (FIG. 3a) shows a shoulder at approximately 47 ppm, corresponding to Gly in an α-helical environment, which decreases after treatment with water and suggests Val in Gly/Val pairs are no longer α-helical. This is an interesting observation in light of recent work into the silks of aquatic spiders, where Gly-Val motifs are common and are suggested to be involved in efficiency of underwater webs.³⁹ Such a response to water by Gly-Val motifs could be a common trigger among these silks. After wet-shearing and drying, a slight collapse in helices occurs from 54% to approximately 51% favoring RC and/or β-sheets, however the predominant structure for Val still remains α-helical. This observation strengthens our prior conclusion that Val residues predominantly exist in well-folded highly-stable α-helical coiled-coil hierarchical structures within spider prey-wrapping silks and conversion to β-sheet due to wetting or wet-shearing is minimal. The high degree of order for Val in α-helical coiled-coil assemblies is further supported by the linewidth of the Val Cβ resonances that are considerably narrower (285 Hz) compared to all other Cβ amino acid line widths (315-510 Hz) in other structures (see Table 1).

### Glutamine

Gln residues are distributed fairly evenly throughout the “W” subunit, with seven in the bead region and two in the linker. In the freshly-spun fibers, 2D DARR data identifies multiple Gln environments including β-sheets, α-helices and disordered structures (FIG. 4a). Low signal to noise makes it difficult to obtain quantitative measurements, but visual inspection of the 2D ¹³C-¹³C DARR SSNMR spectra seems to suggest a fairly even distribution of structures (FIG. 4a, black). After water-wetting, Gln cross peaks look fairly similar with perhaps a slight but non-definitive increase in β-sheet (FIG. 4a, blue). After wet-shearing however, Gln Cα/Cβ contacts become much more resolved and there is a clear bias for Gln Cα/Cβ β-sheet environments over helices (FIG. 4a, red).

### Inter-Residue Contacts:

Several inter-residue cross peaks emerge or become clearer in the 2D ¹³C-¹³C SSNMR correlation spectra after the native silk is cross-linked with water. First, after water treatment a correlation between Ser Cα (57.4 ppm) and Ala Cα (51.4 ppm) in a β-sheet environment is considerably stronger (FIG. 4a, blue). These cross correlations must arise from regions in the silk where Ala and Ser residues are near in space. The DARR 100 ms mixing period applied in these experiments is sufficiently long for ¹³C-¹³C contacts to occur within a single amino acid residue, and in some cases between neighboring amino acids, but not long enough to diffuse between differing domains.⁴⁰ Thus, we safely assume that these observed inter-residue correlations are from amino acid couplets in the primary sequence for AcSp1. Many Ala-Ser/Ser-Ala pairs exist in the W repeat and are concentrated in the aforementioned Ala-Ser/Ser-Ala-rich motifs in the linker domain, but some also are found in the globular helical bead. The simplest and most likely explanation for this emerging inter-residue correlation is that a higher fraction of Ser/Ala-rich motifs adopt β-sheets after the silk is exposed to water. An additional correlation between Gly Cα and Ala Cα becomes more evident after wetting (FIG. 4a, blue). Based on the ¹³C Ala Cα chemical shift (approximately 52.1 ppm), this most likely represents Ala/Gly pairs in disordered structures. Ala/Gly pairs are concentrated in the linker region along with a small grouping located in helix 1. Since helix 1 and the linker are continuous in the primary protein sequence, we suggest that Ala/Gly intermolecular interactions arise from these regions, possibly from different fiber strands. Regardless, the increase in intensity of this inter-residue correlation after water wetting strongly indicates that overall order and rigidity has increased around these Gly/Ala amino acid pairs. However, the inter-residue contact is broad and on the threshold of the noise so we proceed with caution when interpreting these results.

### Domain Sizes in AC Silk

Since β-sheet formation is the dominant mechanism behind water-induced cross-linking of prey-wrapping silk, we next implemented the 2D Wide-Line Separation (WISE) NMR technique with ¹H spin-diffusion to better understand the domain size and overall water-accessibility of these β-sheet nanostructures. The concept behind the technique is outlined elsewhere,⁴¹⁻⁴³ but briefly, by observing the ¹³C chemical shift in the direct dimension and measuring the associated ¹H profile in the indirect dimension, the 2D WISE NMR technique is capable of correlating local biopolymer conformational structure with mobility and dynamics. A 1D ¹H slice is typically extracted from the 2D ¹H/¹³C WISE SSNMR spectrum to reveal the ¹H lineshape at a specific ¹³C chemical shift where broad ¹H lineshapes are associated with “rigid” ¹H environments, while narrow ¹H profiles would arise from more “mobile” regions.

Importantly, if a ¹H spin-diffusion period is included in the WISE experiment it is possible to observe magnetization from the “mobile” water-plasticized regions diffuse into these water-inaccessible domains which ultimately allows for domain-size estimates of the rigid structures.⁴¹ Here we apply the WISE technique to both Black Widow (BW) dragline silk fibers and cross-linked AC silks and compared the respective β-sheet domain sizes and overall fiber rigidity. Work by Holland (2004) was followed; we collected 2D WISE data on D₂O-hydrated isotopically-enriched silks at a series of increasing mixing times and observed the associated ¹H profile of the Ala Cα β-sheet environment.¹⁵ D₂O was used as the plasticizing solvent over H₂O so that the narrow component can be properly attributed to silk polymer protons and not water. The extracted ¹H profiles were fit to both broad and narrow components for each mixing time, and the area of the narrow component was plotted against the square root of the spin-diffusion mixing time.⁴¹ The point (τ*ₘ) at which the narrow component reaches a plateau implies that diffusion of magnetization from the “mobile” domain into the “rigid” domain has reached an equilibrium. Larger domains would require more time to reach equilibrium. This technique has successfully been applied to determine the domain sizes in a number of heterogeneous materials.^{15,41,44}

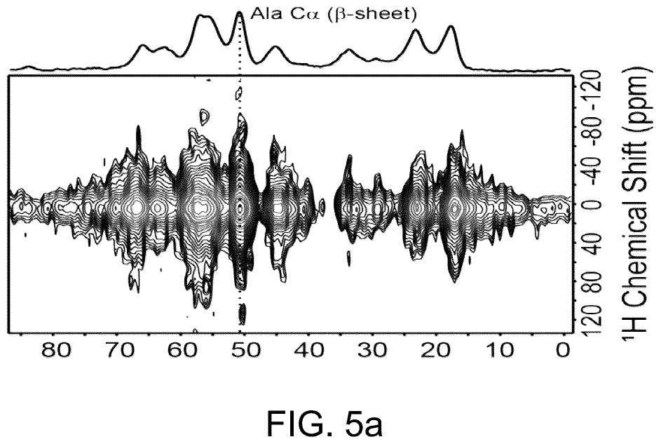

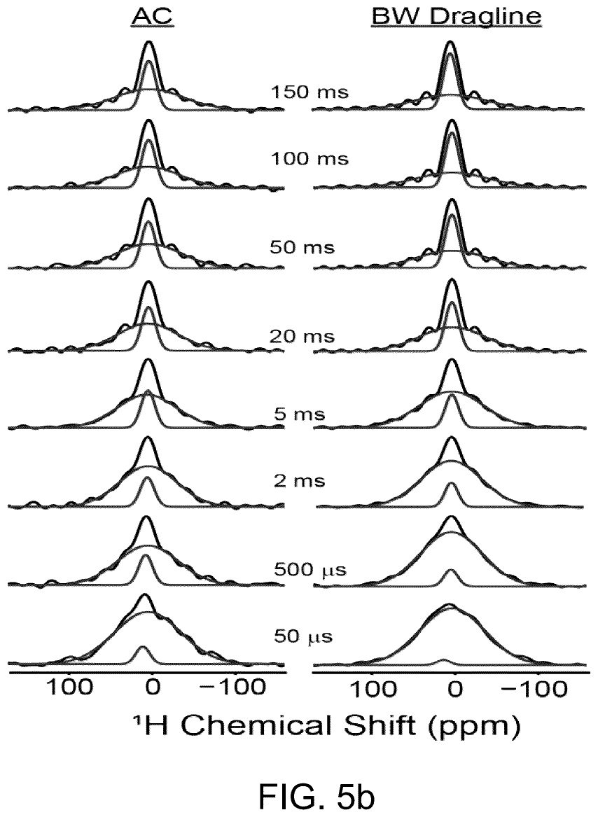

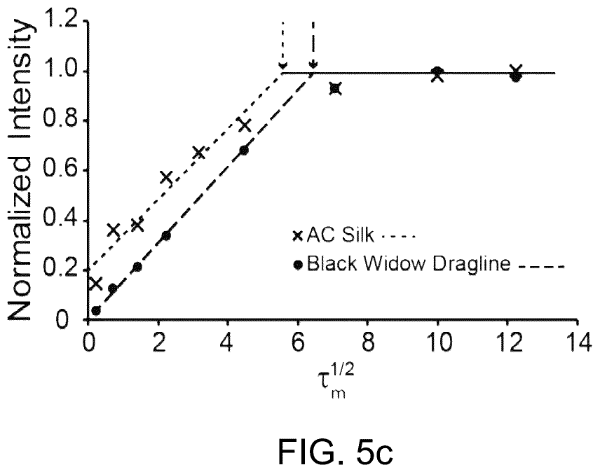

WISE experiments were collected on AC silk and BW dragline fibers using spin-diffusion times between 0.05-150 ms (FIG. 5a). Slices were extracted from WISE experiments at the Ala Cα β-sheet resonance and fit to a broad and narrow component (FIG. 5b). We found the following major conclusions from the WISE buildup data on cross-linked AC silk fibers; 1) a large fraction of Ala residues adopting β-sheet secondary structures within AC silks are inherently water accessible, 2) Gly residues are more dynamic within AC silks compared to BW dragline silks, and 3) water-inaccessible β-sheet nanodomains are significantly smaller in cross-linked AC silks compared to well-characterized BW spider dragline β-sheet nanocrystallites. The first conclusion is clear from the higher initial intensity of the narrow component for AC relative to dragline silk. Even at short (FIGS. 5b, 50 and 500 μs) ¹H spin-diffusion times, the ¹H profile of Ala Cα β-sheet environment from PW silk shows a significantly larger mobile component compared to the corresponding mobile component of the BW dragline silk. This observation implies that even when Ala adopts β-sheet secondary structures in water-hydrated AC silks, a larger fraction of these structures are inherently water-accessible relative to those within BW dragline fibers. Similarly, based on the high initial offset and quick rise to equilibrium, it is clear that Gly residues within AC silks are highly mobile even after water-induced crosslinking (Supplementary Fig. S1). Third, analysis of the Ala C□ spin-diffusion buildup curves for water-hydrated AC and BW dragline silks suggest that the Ala-rich β-sheet domains in cross-linked AC silks are smaller than in BW dragline fibers. We determined the τ*ₘ for AC and BW dragline fibers to be 5.3 and 6.6 ms, respectively (FIG. 5c). Using the previously-derived equation to calculate domain size, we determine the β-sheet domains in cross-linked AC silks to be between 3-7 nm (detail in supplemental). Additionally, we calculated the size of the nanodomains in BW dragline silk to be approximately 11 nm, somewhat larger than reported by XRD results which show a crystallite size of 3.02×4.15×6.71 nm.¹⁶ The difference in size could be due to the well-diffracting, truly crystalline areas of these nanodomains which may be smaller than the water-inaccessible domains we probe with NMR. Obviously it was not possible to measure the initial domain sizes of native AC silks because the technique inherently requires water-hydrating and shearing under MAS. Since overall β-sheet content increases upon water contact and shearing, the original β-sheet nanodomains freshly-spun wrapping silks are either smaller than 3 nm and increase in size upon wetting (unlikely), or more likely there is an increased abundance of small-sized β-sheet domains after water-induced crosslinking.

### CONCLUSIONS

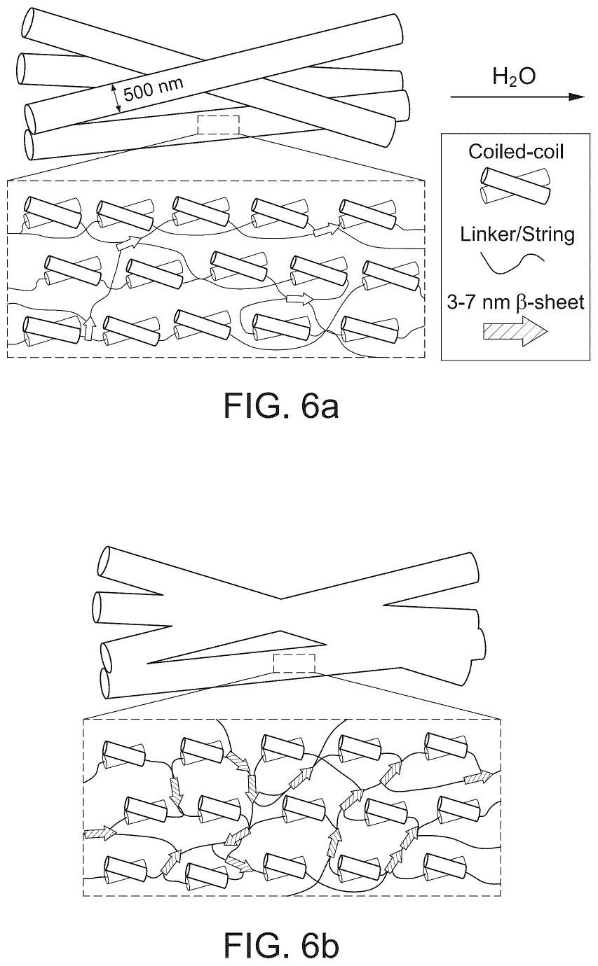

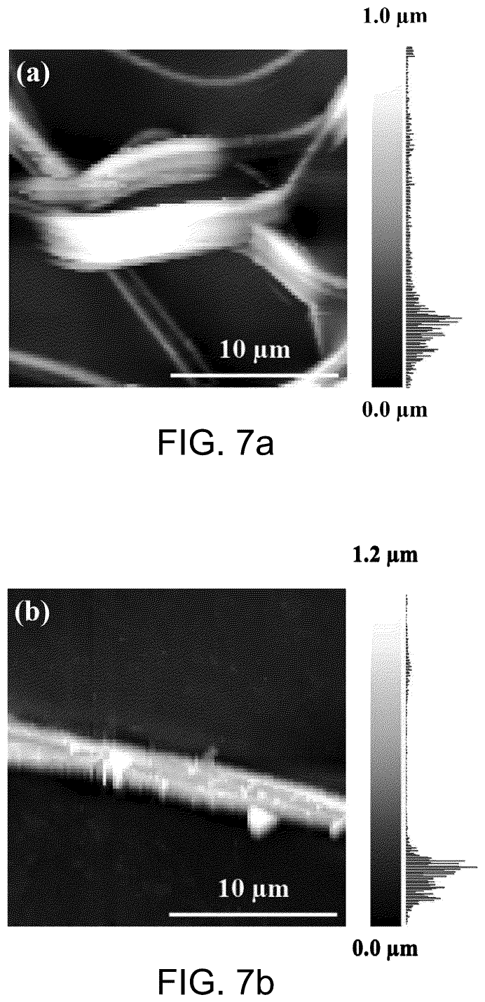

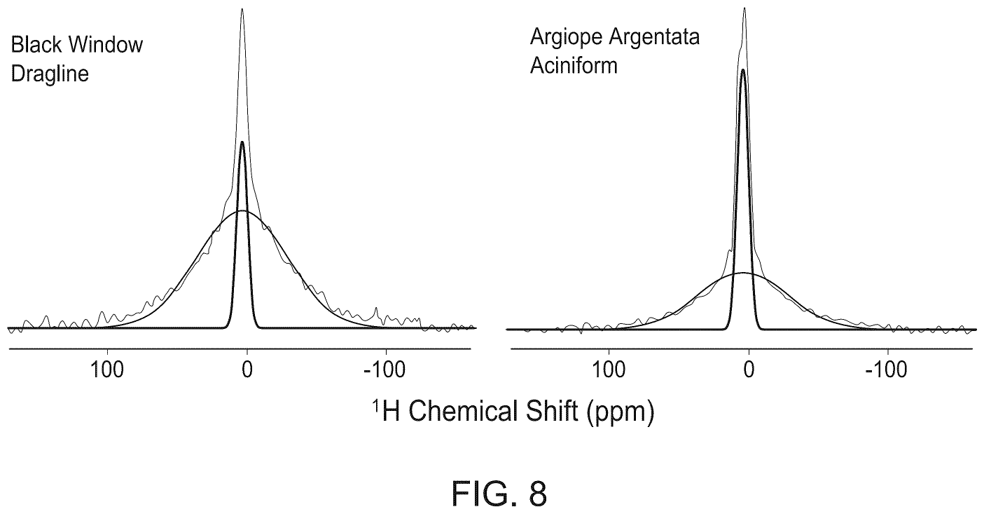

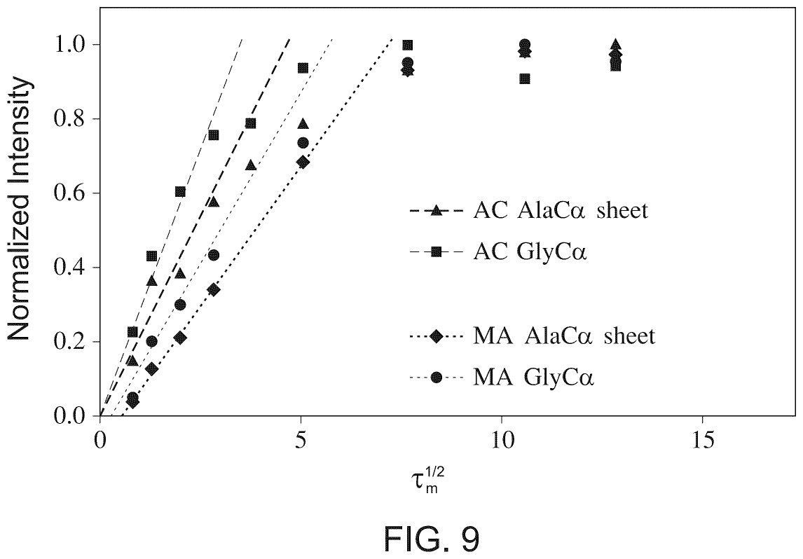

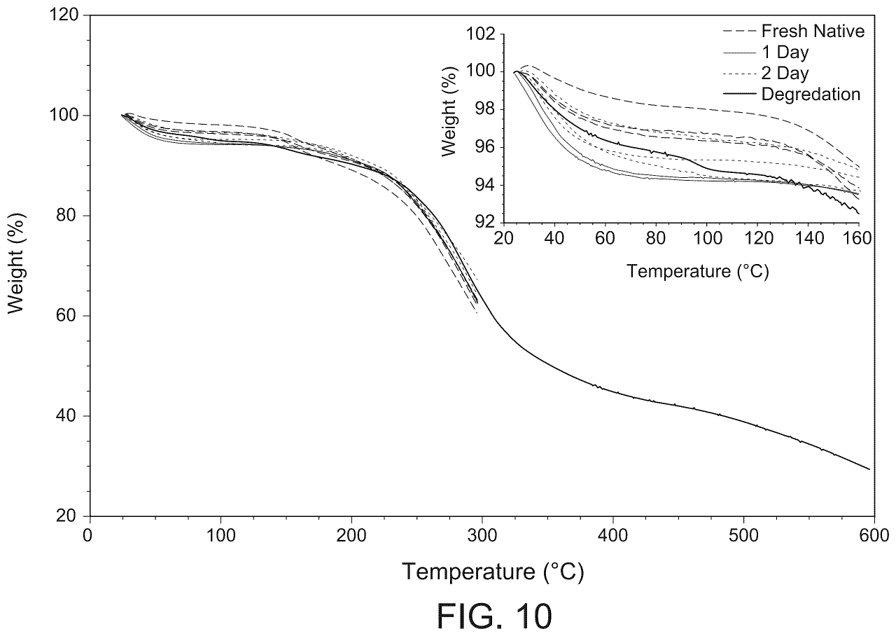

In summary, we have shown that prey-wrapping silks from the garden spider A. argentata undergo a remarkable cross-linking behavior when exposed to moisture. Upon contact with water, SEM images reveal that individual silk fibers fuse together to form cross-linked fibrous sheets and mats. Comprehensive solid-state NMR data on ¹³C-enriched wrapping silks has uncovered the molecular mechanism of inter-fiber cross-linking. Orthogonal isotopic enrichment schemes (Ala-labeling and Val-labeling) were chosen to understand these structural changes for Ala, Ser, Gly, and Val residues, shedding light on which regions of the primary protein sequence are most susceptible to water-induced β-sheet crosslinking. Overall, the data reveals that water-induced fiber cross-linking is driven by an increase in β-sheet protein secondary structure at the expense of disordered and loosely-structured α-helical motifs, whereas well-ordered coiled-coil structures remain in-tact for the most part. However, helical coiled-coil motifs also undergo partial α-to-β conversion with the addition of mechanical shear. This behavior is also seen in other structural protein-based biopolymers. For example, keratin has been shown to convert from α-to-β when stretched in the presence of moisture.^{45,46} Finally, WISE NMR data was used to estimate the β-sheet domain sizes and overall protein dynamics within water-hydrated wrapping silks. Even after water-induced crosslinking, β-sheet regions within AC silks are smaller (approximately 3-7 nm) than those found in BW dragline fibers (approximately 11 nm), suggesting that newly-formed β-sheet structures are small in size and likely between two or more protein chains and potentially involving multiple fibers. FIG. 6 shows a proposed model to illustrate the structural changes of AC silks after water treatment. This data supports our prior conclusions that at a minimum there are three distinct domains in wrapping silks: (1) disordered regions that are easily plasticized by water, (2) rigid α-helices from highly-stable coiled-coil motifs, and (3) Ala and Ser-rich β-sheets are present but surprisingly minimal. Future work will further divulge the role of water in this process, turning a flexible silk fiber into a hardened matted sheet. Utilizing water treatment to convert a protein-based biomaterial from a flexible and extensible fiber dominated by alpha-helical coiled-coil hierarchy to a rigid crossed-linked β-sheet mat provides new avenues in bioinspired material design and applications.

### METHODS

A. Argentata spiders were force-fed isotopically enriched amino acid solutions of either ¹³C-Ala (and Ser, Gly, Gln through metabolism of Ala) or ¹³C-Val. These orthogonal labeling schemes were chosen because Val is almost exclusively found in the helix-rich globular “bead” domain, Ala and Ser residues are dispersed throughout both bead and “linker/string”, Gly is heavily represented in the disordered linker/string region, and Gln is fairly evenly distributed.

Silk samples were imaged on with a FEI Quana 450 FEG Scanning Electron Microscope (SEM) in the Electron Microscope Facility at San Diego State University, using a 10 kV accelerating voltage. Samples were coated with 8 nm of platinum prior to imaging using an EMS 150 Sputter Coater.

Solid-state NMR (SSNMR) experiments were collected with a 600 MHz Bruker AVANCE III HD spectrometer equipped with a 1.9 mm HCN MAS probe. 1D ¹³C CP-MAS NMR experiments were collected at 30 kHz MAS and used a 2.45 μs 90° pulse, 24 ms acquisition time and 3-5 s recycle delay. In order to correctly assign chemical shifts and choose linewidths for peak fitting of 1D data, we extracted these parameters from 2D dipolar-assisted rotational resonance (DARR) experiments. DARR is a 2D homonuclear spin-diffusion technique that correlates nearby ¹³C-¹³C nuclei.⁴⁷ The data is presented in 2D, where the off-diagonal peaks correspond to through-space ¹³C-¹³C interactions. By adjusting the mixing time, one can allow spin-diffusion to occur for longer periods and thus probe longer distances. 2D DARR experiments were collected at 14 kHz MAS with 256 points in the fl dimension with 256 scans, a mixing time of 100 ms and a recycle delay of 2.5 s. During DARR mixing periods, continuous wave ¹H irradiation was applied at a rotary-resonance (ωᵣ=ω_{rf}) of 14 kHz.

WISE experiments were collected at 5 kHz MAS with a 300 s contact time and 5 s recycle delay. All spectra were referenced to DSS at 0.0 ppm by setting the downfield adamantane resonance to 40.49 ppm.³⁶ Samples were soaked in D₂O before experiments. The equation below is used to calculate the β-sheet domain size, d_{dis}, for the silks:

$${d}_{dis} = \frac{{ρ}_{A}^{H} {ϕ}_{A} + {ρ}_{B}^{H} {ϕ}_{B}}{{ϕ}_{A} {ϕ}_{B}} \frac{\sqrt{{D}_{A} {D}_{B}}}{{ρ}_{A}^{H} \sqrt{{D}_{A}} + {ρ}_{B}^{H} \sqrt{{D}_{B}}} \frac{4 ε {ϕ}_{dis}}{\sqrt{π}} \sqrt{{τ}_{m}^{*}} \qquad ( 1 )$$

where ρ^{H}, ϕ_{dis}, D and ε are the proton density of the two domains, volume fraction of the two domains, ¹H spin-diffusion coefficients for both domains, and dimensionality, respectively.⁴¹ A summary of the coefficient values used for our calculations and explanation is in the Supplemental. All silk materials were center-packed with Teflon.

### WISE Calculations for Domain Size

Equation 1 below is used to calculate the β-sheet domain size, d_{dis}, for the silks:

$${d}_{dis} = \frac{{ρ}_{A}^{H} {ϕ}_{A} + {ρ}_{B}^{H} {ϕ}_{B}}{{ϕ}_{A} {ϕ}_{B}} \frac{\sqrt{{D}_{A} {D}_{B}}}{{ρ}_{A}^{H} \sqrt{{D}_{A}} + {ρ}_{B}^{H} \sqrt{{D}_{B}}} \frac{4 ε {ϕ}_{dis}}{\sqrt{π}} \sqrt{{τ}_{m}^{*}} \qquad ( 1 )$$

where ρ^{H}, ϕ_{dis}, D and & are the proton density of the two domains, volume fraction of the two domains, ¹H spin-diffusion coefficients for both domains, and dimensionality, respectively.¹ The value of t*ₘ for all silks was extracted from the build-up curve of the narrow component of the data by fitting the Ala Cα resonance with a broad and narrow component (Figure S2). The linewidths in for BW were 47 kHz for the broad, and 5.2 kHz for the narrow. In AC fibers, the linewidths were 45 kHz for the broad, and 5 kHz for the narrow. We assume the proton densities for AC β-sheets are similar to those in BW dragline. The proton densities for β-sheets domains and disordered regions in dragline are 0.107 and 0.078 g/cm³, respectively. The spin-diffusion coefficient can be determined by the ¹H linewidth (Δv_{1/2}) and average minimum proton distance (r_{HH}) with the following proportionality: D˜Δv_{1/2}(r_{HH})² (Clauss et al Acta Polymer 1993, 44, 1). The spin-diffusion of PS-PMMA is known to be approximately 0.8 nm²/ms with a ¹H linewidth of 38 kHz (Clauss et al Acta Polymer 1993, 44, 1). Using this proportionality, we estimate the spin-diffusion coefficients for the dynamic and rigid regions of water-treated AC silks to be 0.11 and 0.95 nm2/ms, respectively. The crystalline domain volume of BW dragline fibers were recently determined by X-ray scattering and reported to be 40% (Jenkins et al Biomacromolecules 2013, 14, 3472). Because there is no XRD data reported for AC silk the morphology of the β-sheet regions is unknown. However, we assume spin-diffusion can occur into the domains predominantly on four faces, giving it a dimensionality of 2 (ϕ_{dis}=2) based on the location of the β-sheet regions with respect to the rest of the matrix (Addison et al Chem Commun 2018, 54, 10746). The β-sheet fraction for native, as-spun AC fibers was determined from SSNMR in our recent work and is reported to be approximately 15% (Addison et al Chem Commun 2018, 54, 10746). However, we acknowledge that during the WISE experiment the silk may become sheared in the presence of D₂O which could have an impact on the β-sheet content. In the wet-sheared and dried sample, we see an increase in β-sheet from Ala, Val and Ser although deconvolution of Ser is difficult because of spectral overlap as discussed in the main text. Using an approach similar to our previous work, we use the primary sequence and secondary structure for Ala, Val and Ser to roughly assign total β-sheet content (Addison et al Chem Commun 2018, 54, 10746), and estimate an upper-limit of the β-sheet fraction to be 25% in the wet-sheared and dried material. With this in mind, the calculated size of these crystallites using a β-sheet fraction between 15-25% produces a range of nanocrystallite sizes between 4.4-6.4 nm. Table S1 summarizes the parameters used for calculating the domain sizes in both Black Widow (L. hesperus) dragline MA and A. argentata AC silks.

TABLE S1
Summary of parameters used to calculate domain size in BW
dragline and AC silks.

| | Major | Aciniform | Aciniform |
|---|---|---|---|
| Parameter | Ampullate | (lower limit) | (upper limit) |
| P_{A}^{H} (g/cm²) | 0.107 | 0.107 | 0.107 |
| P_{B}^{H} (g/cm²) | 0.078 | 0.078 | 0.078 |
| ϕ_{A} | 0.40 | 0.15 | 0.25 |
| ϕ_{B} | 0.60 | 0.85 | 0.75 |
| D_{A} (nm²/ms) | 0.99 | 0.95 | 0.95 |
| D_{B} (nm²/ms) | 0.11 | 0.11 | 0.11 |
| ε | 2 | 2 | 2 |
| \sqrt{τ*ₘ} | 6.6 | 4.5 | 4.5 |
| \sqrt{τ*ₘ} Error | ±0.3 | ±0.5 | ±0.5 |
| d_{dis} (nm) | 11.0 ± 0.6 | 4.9 ± 0.5 | 5.8 ± 0.6 |

TABLE S2
Summary of parameters used to calculate domain size in L. hesperus MA silks
and A. argentata AC silks for both Gly Cα and Ala Cα.

| | | AC | AC | | AC | AC |
|---|---|---|---|---|---|---|
| | MA | Ala Cα | Ala Cα | MA | Gly Cα | Gly Cα |
| Parameter | Ala Cα | (LL) | (UL) | Gly Cα | (LL) | (UL) |
| ρ_{A}^{H} | 0.107 | 0.107 | 0.107 | 0.107 | 0.107 | 0.107 |
| (g/cm²) | | | | | | |
| ρ_{B}^{H} (g/cm²) | 0.078 | 0.078 | 0.078 | 0.078 | 0.078 | 0.078 |
| ϕ_{A} | 0.40 | 0.15 | 0.25 | 0.40 | 0.15 | 0.25 |
| ϕ_{B} | 0.60 | 0.85 | 0.75 | 0.60 | 0.85 | 0.75 |
| D_{A} | 0.99 | 0.95 | 0.95 | 0.99 | 0.95 | 0.95 |
| (nm²/ms) | | | | | | |
| D_{B} | 0.11 | 0.11 | 0.11 | 0.11 | 0.11 | 0.11 |
| (nm²/ms) | | | | | | |
| E | 2 | 2 | 2 | 2 | 2 | 2 |
| \sqrt{τₘ*} | 6.6 | 4.5 | 4.5 | 5.1 | 2.9 | 2.9 |
| \sqrt{τₘ*} Error | ±0.3 | ±0.5 | ±0.5 | ±0.5 | ±0.3 | ±0.5 |
| d_{dis} (nm) | 11.0 ± 0.6 | 4.9 ± 0.5 | 5.8 ± 0.6 | 8.6 ± 0.8 | 3.1 ± 0.4 | 3.7 ± 0.6 |

LL: Lower limit, UL: Upper-limit

The results presented above illustrate that narrow ¹H components (disordered, non-β-sheet) spin diffuse more rapidly into broad components for Gly compared to Ala. This is the case for both AC and MA silks albeit the length scale are shorter for AC silk. In both cases, this implicates that Gly is spatially located at the periphery of the β-sheet (interphase). For, MA silks the interpretation would be that the poly(Ala) is the core of the β-sheet and the flanking Gly-Ala units are β-sheet but located at the β-sheet interface thus, mobile regions in the disordered (amorphous) phase are closer to Gly than Ala. It appears to be something similar in AC silk where for the most part Ala is located in the β-sheet core with Gly-containing units closer to the β-sheet interface with the more disordered or α-helical regions. It should be noted that this is speculative for AC silk however, the result is consistent with our previous studies for MA silk (G. P. Holland, R. V. Lewis, J. L. Yarger, J Am Chem Soc 2004, 126, 5867).

TABLE S3
Calculated water loss for prey-wrapping silk samples from FIG. S3,
their average and standard deviation. Native samples were run within
5 minutes of collection. AC silk samples were soaked in DI water for
2 hours and dried at 25° C. at a humidity of 30-35% for 1 or 2 days.

| Sample | Weight Loss (%) | Water Content Average ± SD |
|---|---|---|
| Native 1 | 2.359 | 3.2 ± 0.6 |
| Native 2 | 3.778 | |
| Native 3 | 3.413 | |
| 1 Day, 1 | 5.704 | 5.76 ± 0.05 |
| 1 Day, 2 | 5.813 | |
| 2 Day, 1 | 4.729 | 4.7 ± 0.7 |
| 2 Day, 2 | 5.645 | |
| 2 Day, 3 | 3.825 | |

### Example 2: Exemplary Wet-Shearing/Solvent-Shearing Method for Aciniform (AC) Silk Processing

This example demonstrates exemplary methods for making hydrated, or hydrated and dried, orb-weaving spider aciniform (AC) prey-wrapping silks, or orb-weaving spider aciniform (AC) prey-wrapping silks that have been substantially converted into β-sheet aggregates or β-sheet nanocrystallite structures, and products of manufacture comprising same.

This exemplary process for wet-shearing or solvent-shearing AC silk fibers comprises:
(1) the silk sample is soaked in water (H₂O) or a solvent for a time period (for example, soaked mins to days, optionally between about 4 minutes to 60 minutes, or between about 1 hour to 24 hours, or between about a day to one week),
(2) excess water or solvent is removed with a KIMWIPE™ or equivalent, wherein optionally between about 50% to 99% of the water and/or solvent is removed, or about 80%, 85%, 90%, 91%, 92%, 93%, 94%, 95%, 96%, 97%, 98%, or 99%, or more of the water and/or solvent is removed, and
(3) the “wet” or “solvated” silk sample is transferred to a magic angle spinning (MAS) solid-state nuclear magnetic resonance (NMR) rotor for mechanical wet-shearing or solvent-shearing (using protocols described for example, in Andrew, E. R., et al., Nuclear Magnetic Resonance Spectra from a Crystal Rotated at High Speed. Nature 1958, vol. 182, pg 1659; Zhang, R., et al., Proton-based Ultrafast Magic Angle Spinning Solid-state NMR Spectroscopy. Acc. Chem. Res. 2017, vol. 50, pg 1105).

The MAS rotor is spun about the magic angle (54.7°) with respect to the NMR spectrometer external magnetic field, B₀ (see FIG. 1). The MAS speed or rotor frequency, ν_{R} (Hz; s⁻¹), is precisely controlled with a MAS pneumatic spin control unit (Bruker Biospin). Various rotor diameters can be chosen that dictate the maximum rotor frequency and can range from 1 kHz to 100 KHz which depends on the rotor diameter. Rotor diameters can be varied from 0.5 mm to 10 mm and the spinning speed can be controlled to less than (<) 1%. The MAS NMR probe also has a temperature control unit to precisely control sample temperature.

Although water is chosen to be the solvent for wetting or solvating the silk, it does not need to be, and different solvents could have an impact on the degree of shearing and the molecular structure of the final processed silk product and resulting mechanical properties. Soaking time in H₂O (or solvent), length of time under MAS (degree of shearing), ν_{R}, and temperature are all believed to impact the structure and mechanical properties of the final processed AC silk material. It is hypothesized that the act of dehydrating (drying) the solvent-soaked silk under shear controls the final material structure (β-sheet content) and mechanical properties (stiffness). This method of wet-shearing (including optionally solvent-shearing) the silk within a MAS NMR probe allows for determination of changes in the molecular structure (protein-folding) of the silk during processing via MAS NMR spectroscopy. We have illustrated that the act of wet-shearing or solvent-shearing hydrated or solvated AC silk results in a substantial increase in β-sheet content which impacts the mechanical properties of the material.

### REFERENCES EXAMPLE 1

1 Blackledge, T. A. et al. Sequential origin in the high performance properties of orb spider dragline silk. Sci Rep 2, 782, doi:10.1038/srep00782 (2012).
2 Vollrath, F. et al., Liquid crystalline spinning of spider silk. Nature 410, 541-548, doi:10.1038/35069000 (2001).
3 Vepari, C. et al., Silk as a Biomaterial. Prog Polym Sci 32, 991-1007, doi:10.1016/j.progpolymsci.2007.05.013 (2007).
4 Aigner, T. B., et al., Biomedical Applications of Recombinant Silk-Based Materials. Adv Mater 30, e1704636, doi:10.1002/adma.201704636 (2018).
5 Andersson, M. et al. Biomimetic spinning of artificial spider silk from a chimeric minispidroin. Nat Chem Biol 13, 262-264, doi:10.1038/nchembio.2269 (2017).
6 Vasanthavada, K. et al. Aciniform spidroin, a constituent of egg case sacs and wrapping silk fibers from the black widow spider Latrodectus hesperus. J Biol Chem 282, 35088-35097, doi:10.1074/jbc.M705791200 (2007).
7 Romer, L. et al., The elaborate structure of spider silk: structure and function of a natural high performance fiber. Prion 2, 154-161, doi:10.4161/pri.2.4.7490 (2008).
8 Yarger, J. L., et al., Uncovering the structure-function relationship in spider silk. Nature Reviews Materials 3, doi:10.1038/natrevmats2018.8 (2018).
9 Hayashi, C. Y., et al., Molecular and mechanical characterization of aciniform silk: uniformity of iterated sequence modules in a novel member of the spider silk fibroin gene family. Mol Biol Evol 21, 1950-1959, doi:10.1093/molbev/msh204 (2004).
10 Blackledge, T. A. et al., Silken toolkits: biomechanics of silk fibers spun by the orb web spider Argiope argentata (Fabricius 1775). J Exp Biol 209, 2452-2461, doi:10.1242/jeb.02275 (2006).
11 Xu, M. & Lewis, R. V. Structure of a protein superfiber: Spider dragline silk. Proc Natl Acad Sci U S A 87, 7120-7124 (1990).
12 Ayoub, N. A., et al., Blueprint for a high-performance biomaterial: full-length spider dragline silk genes. PLOS One 2, e514, doi:10.1371/journal.pone.0000514 (2007).
13 Xu, D., et al., Exploring the backbone dynamics of native spider silk proteins in Black Widow silk glands with solution-state NMR spectroscopy. Polymer 55, 3879-3885, doi:10.1016/j.polymer.2014.06.018 (2014).
14 Xu, D. et al. Protein secondary structure of Green Lynx spider dragline silk investigated by solid-state NMR and X-ray diffraction. Int J Biol Macromol 81, 171-179, doi:10.1016/j.ijbiomac.2015.07.048 (2015).
15 Holland, G. P., Lewis, R. V. & Yarger, J. L. WISE NMR Characterization of Nanoscale Heterogeneity and Mobility in Supercontracted Nephila clavipes Spider Dragline Silk. JACS 126 (2004).
16 Jenkins, J. E. et al. Characterizing the Secondary Protein Structure of Black Widow Dragline Silk Using Solid-State NMR and X-ray Diffraction. Biomacromolecules 14, 3472-3483, doi:10.1021/bm400791u (2013).
17 Yang, Z., et al., Small-Angle X-ray Scattering of Spider Dragline Silk. Macromolecules 30, 8254-8261 (1997).
18 Riekel, C. et al. Aspects of X-ray diffraction on single spider fibers. Biological Macromolecules 24, 179-186 (1999).
19 Gray, G. M. et al. Secondary Structure Adopted by the Gly-Gly-X Repetitive Regions of Dragline Spider Silk. Int J Mol Sci 17, doi:10.3390/ijms17122023 (2016).
20 Chaw, R. C. et al. Intragenic homogenization and multiple copies of prey-wrapping silk genes in Argiope garden spiders. BMC Envolutionary Biology 14 (2014).
21 Sarker, M. et al. Tracking Transitions in Spider Wrapping Silk Conformation and Dynamics by ¹⁹F Nuclear Magnetic Resonance Spectroscopy. Biochemistry 55, 3048-3059, doi:10.1021/acs.biochem.6b00429 (2016).
22 Tremblay, M. L. et al. Spider wrapping silk fibre architecture arising from its modular soluble protein precursor. Sci Rep 5, doi:10.1038/srep11502 (2015).
23 Xu, L., et al., ¹H, ¹³C and 15N NMR assignments of the aciniform spidroin (AcSp1) repetitive domain of Argiope trifasciata wrapping silk. Biomol NMR Assign 6, 147-151, doi:10.1007/s12104-011-9344-z (2012).
24 Tremblay, M. L., Xu, L., Sarker, M., Liu, X. Q. & Rainey, J. K. Characterizing Aciniform Silk Repetitive Domain Backbone Dynamics and Hydrodynamic Modularity. Int J Mol Sci 17, doi:10.3390/ijms17081305 (2016).
25 Xu, L., Weatherbee-Martin, N., Liu, X. Q. & Rainey, J. K. Recombinant Silk Fiber Properties Correlate to Prefibrillar Self-Assembly. Small 15, e1805294, doi:10.1002/smll.201805294 (2019).
26 Xu, L. et al. Structural and Mechanical Roles for the C-Terminal Nonrepetitive Domain Become Apparent in Recombinant Spider Aciniform Silk. Biomacromolecules 18, 3678-3686, doi:10.1021/acs.biomac.7b01057 (2017).
27 Xu, L., et al., Recombinant minimalist spider wrapping silk proteins capable of native-like fiber formation. PLOS One 7, e50227, doi:10.1371/journal.pone.0050227 (2012).
28 Addison, B. et al. Spider prey-wrapping silk is an alpha-helical coiled-coil/beta-sheet hybrid nanofiber. Chem Commun (Camb) 54, 10746-10749, doi:10.1039/c8cc05246h (2018).
29 Rousseau, M. E., et al., Conformation and Orientation of Proteins in Various Types of Silk Fibers Produced by Nephila clavipes Spiders. Biomacromolecules 10, 2945-2953 (2009).
30 Lefevre, T., et al., Diversity of molecular transformations involved in the formation of spider silks. J Mol Biol 405, 238-253, doi:10.1016/j.jmb.2010.10.052 (2011).
31 Robinson, M. H. Predatory Behavior of Argiope argentata (Fabricius). Am. Zoologist 9, 161-173 (1969).
32 Eisner, T. et al., Ploy and counterploy in predator-prey interactions: Orb-weaving spiders versus bombardier beetles. Proc Natl Acad Sci U S A 73, 1365-1367 (1976).
33 Eberhard, W. G., et al., The mystery of how spiders extract food without masticating prey. Bull. Br. arachnol. Soc. 13, 372-376 (2006).
34 Asakura, T., et al., Conformational change of ¹³C-labeled 47-mer model peptides of Nephila clavipes dragline silk in poly(vinyl alcohol) film by stretching studied by ¹³C solid-state NMR and molecular dynamics simulation. Int J Biol Macromol 131, 654-665, doi:10.1016/j.ijbiomac.2019.03.112 (2019).
35 Holland, G. P., et al., Quantifying the fraction of glycine and alanine in beta-sheet and helical conformations in spider dragline silk using solid-state NMR. Chem Commun (Camb), 5568-5570, doi:10.1039/b812928b (2008).
36 Morcombe, C. R. et al., Chemical shift referencing in MAS solid state NMR. Journal of Magnetic Resonance 162, 479-486, doi:10.1016/s1090-7807(03)00082-x (2003).
37 Addison, J. B. et al. Structural characterization of nanofiber silk produced by embiopterans (webspinners). RSC Adv 4, 41301-41313, doi:10.1039/C4RA07567F (2014).
38 Addison, J. B. et al. b-Sheet Nanocrystalline Domains Formed From Phosphorylated Serine-rich Motifs in Caddisfly Larval Silk: A Solid State NMR and XRD Study. Biomacromolecules 14, 1140-1148, doi:10.1021/bm400019d (2013).
39 Correa-Garhwal, S. M. et al. Spidroins and Silk Fibers of Aquatic Spiders. Sci Rep 9, 13656, doi:10.1038/s41598-019-49587-y (2019).
40 Hong, M. et al., Magic-Angle-Spinning NMR Techniques for Measuring Long-Range Distances in Biological Macromolecules. Acc Chem Res 46, 2154-2163 (2013).
41 Clauss, J., et al., Determination of domain sizes in heterogeneous polymers by solid-state NMR. Acta Polymer 44, 1-17 (1993).
42 Schmidt-Rohr, K., et al., Correlation of Structure, Mobility, and Morphological Information in Heterogeneous Polymer Materials by Two-Dimensional Wideline-Separation NMR Spectroscopy. Macromolecules 25, 3273-3277 (1992).
43 K. Schmidt-Rohr, H. W. S. Multidimensional Solid-State NMR and Polymers. 1 edn, (Academic Press, 1994).
44 Yan, B. & Stark, R. E. A WISE NMR Approach to Heterogeneous Biopolymer Mixtures: Dynamics and Domains in Wounded Potato Tissues. Macromolecules 31, 2600-2605 (1998).
45 Kreplak, L. et al. A New Deformation Model of Hard α-Keratin Fibers at the Nanometer Scale: Implications for Hard α-Keratin Intermediate Filament Mechanical Properties. Biophysical Journal 82, 2265-2274 (2002).
46 Kreplak, L., et al., New Aspects of the a-Helix to b-Sheet Transition in Stretched Hard a-Keratin Fibers. Biophys J 87, 640-647, doi:10.1529/biophysj. 103.036749 (2004).
47 Takegoshi, K., et al., ¹³C-¹H dipolar-assisted rotational resonance in magic-angle spinning NMR. Chemical Physics Letters 344, 631-637 (2001).

A number of embodiments of the invention have been described. Nevertheless, it can be understood that various modifications may be made without departing from the spirit and scope of the invention. Accordingly, other embodiments are within the scope of the following claims.

1. A method for making a biomaterial comprising: providing an orb-weaving spider aciniform (AC) prey-wrapping silk; and exposing the orb-weaving spider aciniform (AC) prey-wrapping silk to solvent, an aqueous solution or to water, to: partially or substantially hydrate the orb-weaving spider aciniform (AC) prey-wrapping silk, and/or, to substantially convert silk alpha helices into silk β-sheet aggregates.

2. The method of claim 1, wherein approximately 1% to 99%, or at least 5% by weight of the hydrated orb-weaving spider aciniform (AC) prey-wrapping silk is the water or aqueous solution or solvent.

3. The method of claim 1, wherein the orb-weaving spider aciniform (AC) prey-wrapping silk is water-wetted or hydrated or solvated for between about 10 minute to 10 hours or more.

4. The method of claim 1, wherein the hydrated or solvated orb-weaving spider aciniform (AC) prey-wrapping silk is dried.

5. The method of claim 4, wherein the substantially or partially dried orb-weaving spider aciniform (AC) prey-wrapping silk has a substantially β-sheet nanocrystallite structure.

6. The method of claim 1, wherein approximately 5% to 99%, or at least about 85% or more of the orb-weaving spider aciniform (AC) prey-wrapping silk alpha helices are converted into silk β-sheet aggregates or β-sheet nanocrystallite structures.

7. The method of claim 1, wherein the solvent comprises a water-miscible protonic and/or a polar aprotonic organic solvent.

8. A composition or a product of manufacture comprising a biomaterial made by a method claim 1.

9. A composition or a product of manufacture comprising a plurality of silk β-sheet aggregates or β-sheet nanocrystallite structures, made by process comprising a method claim 1.

10. The composition or a product of manufacture of claim 9, wherein the composition or a product of manufacture comprises or is fabricated as a film, a thread or a fiber, and optionally the thread or fiber is woven or fabricated into a cloth, textile or a fabric, or a wearable material, or silk β-sheet aggregates or β-sheet nanocrystallite structures are incorporated into a plastic, a wood, a synthetic material, or a cloth.

11. A product of manufacture comprising a biomaterial comprising orb-weaving spider aciniform (AC) prey-wrapping silk, or silk β-sheet aggregates and/or β-sheet nanocrystallite structures, wherein approximately 5% to 99%, or 10% to 90%, or 15% to 85% or 20% to 80%, or at least about 85%, 90%, 91%, 92%, 93%, 94%, 95%, 96%, 97%, 98%, or 99% or more, of the orb-weaving spider aciniform (AC) prey-wrapping silk alpha helices have been converted into silk β-sheet aggregates and/or β-sheet nanocrystallite structures.

12. The product of manufacture of claim 11, fabricated as a thread or fiber, and optionally the thread or fiber is woven or fabricated into a cloth, textile or a fabric.

13. The product of manufacture of claim 11, fabricated as a container or a medical device, optionally a stent, or a bandage.

14. The product of manufacture of claim 11, fabricated as an adsorbant or absorbant material or an insulating material.

15. The product of manufacture of claim 11, fabricated as or incorporated into a building material, a structural material or a construction material, optionally comprising or incorporated into or a part of: an automobile, an airplane, a spacecraft, a boat, a dwelling or office or commercial building, or for use or incorporation into or as walls, ceilings or roofs, insulating materials, lining materials, ducts, piping, wiring insulation, wall or ceiling coverings, panels, sinks, or floors.

16. The method of claim 1, wherein approximately 5% to 90%, or 10% to 80%, or 20% to 70%, or at least 10%, 15%, 20%, or 55%, by weight of the hydrated orb-weaving spider aciniform (AC) prey-wrapping silk is the water or aqueous solution or solvent.

17. The method of claim 3, wherein the orb-weaving spider aciniform (AC) prey-wrapping silk is water-wetted or hydrated or solvated for between about 10 minute to 10 hours, or for about 2 hours (hr).

18. The method of claim 4, wherein the hydrated or solvated orb-weaving spider aciniform (AC) prey-wrapping silk is dried for between about 1 hour and 5 days, or for between about 10 hours and 3 days, or more, or for about 2 days.

19. The method of claim 6, wherein approximately 10% to 90%, or 15% to 85% or 20% to 80%, or at least about 90%, 91%, 92%,, 93%, 94%, 95%, 96%, 97%, 98%, or 99%, or more of the orb-weaving spider aciniform (AC) prey-wrapping silk alpha helices are converted into silk β-sheet aggregates or β-sheet nanocrystallite structures.

20. The method of claim 7, wherein:
(a) the solvent comprises an alcohol or a glycol, and optionally the alcohol or glycol comprises ethylene glycol, methanol, propanol, butanol, isopropanol, isobutanol, furfuryl alcohol, 1,2-butanediol, 1,3-butanediol, 1,4-butanediol, 2-butoxyethanol, and/or hexafluoroisopropanol,
(b) the solvent comprises an acid or is an acid solution, and optionally the acid comprises phosphoric acid, formic acid, butyric acid, trinitrobenzenesulfonic acid (TNBS) and/or acetic acid,
(c) the solvent comprises an inorganic salt, and optionally the inorganic salt comprises a halide and/or a thiocyanate, and/or,
(d) the solvent comprises dimethyl sulfoxide, 1, 4 dioxane, ethylamine, N-methyl-2-pyrrolidone, glycerol, acetone, acetaldehyde, diethanolamine, diethylenetriamine, dimethylformamide, dimethoxyethane, triethylene glycol, tetrahydrofuran and/or acetonitrile


SEQUENCE LISTING (6 sequences)

SEQ ID NO: 1 - protein, 59 aa, Argiope argentata
AGPQGGFGATGGASAGLISRVANALANTSTLRTVLRTGVSQQIASSVQRAAQSLASTLG

SEQ ID NO: 2 - protein, 17 aa, Argiope argentata
VDGNNLARFAVQAVSRL

SEQ ID NO: 3 - protein, 23 aa, Argiope argentata
PAGSDTSAYAQAFSSALFNAGVL

SEQ ID NO: 4 - protein, 28 aa, Argiope argentata
NASNIDTLGSRVLSALLNGVSSAAQGLG

SEQ ID NO: 5 - protein, 19 aa, Argiope argentata
INVDSGSVQSDISSSSSFL

SEQ ID NO: 6 - protein, 51 aa, artificial sequence
  synthetic peptide
STSSSSASYSQASASSTSGSGYTGPSGPSTGPGYPGPLGGGAPFGQSGFGG